(19) United States
(12) Patent Application Publication
(10) Pub. No.: US 2013/0109932 A1
(43) Pub. Date: May 2, 2013

(54) SYSTEMS AND METHODS FOR PULMONARY MONITORING AND TREATMENT

(71) Applicant: Sonitus Medical, Inc., San Mateo, CA (US)

(72) Inventors: Vahid SAADAT, Atherton, CA (US); Amir A. ABOLFATHI, Woodside, CA (US)

(73) Assignee: SONITUS MEDICAL, INC., San Mateo, CA (US)

(21) Appl. No.: 13/715,291

(22) Filed: Dec. 14, 2012

Related U.S. Application Data
(63) Continuation of application No. 12/116,860, filed on May 7, 2008.

(51) Int. Cl. A61B 5/0205; A61B 5/00; A61B 7/00; A61B 5/0408; A61B 5/01; A61B 5/087; A61B 5/0478; A61B 5/08; A61B 5/0402

(52) U.S. Cl. CPC A61B 5/02055; A61B 5/08; A61B 5/682; A61B 7/003; A61B 5/082; A61B 5/7405; A61B 5/7455; A61B 5/74; A61B 5/4818; A61B 5/0205; A61B 5/0402; A61B 5/746; A61B 5/4839; A61B 5/01; A61B 5/087; A61B 5/0873; A61B 5/0878; A61B 5/0088; A61B 5/0478; A61B 5/0408; A61B 5/6839; A61B 5/6832; A61B 5/6834; A61B 5/6831
USPC 600/301; 600/529; 600/532; 600/484; 600/538; 600/476; 600/383

(57) ABSTRACT

Systems and methods are disclosed determining a pulmonary function by mounting one or more sensors intra-orally; capturing intra-oral data; and determining the pulmonary function based on an analysis of the intra-oral data.

### CROSS-REFERENCE TO RELATED APPLICATIONS

This application is a continuation of U.S. patent application Ser. No. 12/116,80 filed May 7, 2008, which is incorporated herein by reference in its entirety.

### BACKGROUND

Pulmonary diseases and disorders continue to pose major health care concerns. As discussed in U.S. Pat. No. 7,329,226, diseases and disorders are of either an obstructive or restrictive nature. Obstructive breathing diseases are caused by a blockage or obstacle is the airway due to injury or disease, such as asthma, chronic bronchitis, emphysema, or advanced bronchiectasis. Restrictive breathing disorders are caused by muscular weakness, a loss of lung tissue or when lung expansion is limited, as a result of decreased compliance of the lung or thorax. The conditions that can result in a restrictive breathing disorder include pectus excavatum, myasthenia gravis, diffuse idiopathic interstitial fibrosis, and space occupying lesions, such as tumors and effusions. Proper treatment of pulmonary diseases and disorders requires early identification and on-going monitoring of pulmonary performance.

As noted in the '226 patent, conventionally, pulmonary performance is tested in a clinical setting to establish certain baseline values indicative of the ability of the lungs to exchange oxygen and carbon dioxide during normal breathing. Pulmonary performance can be established by testing pulmonary volumes using a spirometer during inspiration and expiration, as measured under normal and forced conditions. Spirometric testing can determine tidal volume, which is the volume inhaled or exhaled in normal quiet breathing; inspiratory reserve volume (IRV), which is the maximum volume that can be inhaled following a normal quiet inhalation; expiratory reserve volume (ERV), which is the maximum volume that can be exhaled following a normal quiet exhalation; and inspiratory capacity (IC), which is the maximum volume that can be inhaled following a normal quiet exhalation

In addition, functional residual capacity (FRC), which is the volume remaining in the lungs following a normal quiet exhalation, can be measured by introducing helium into a closed spirometer at the end of a normal quiet exhalation and determining FRC from helium concentration upon reaching equilibrium. However, for patients suffering from obstructive respiratory disorders, such as emphysema, the helium dilution technique can underestimate FRC. Alternatively, FRC can also be measured through body plethysmography.

Pulmonary performance testing in a non-clinical setting is difficult. Testing requires the same equipment as required in-clinic. Moreover, ensuring that the battery of pulmonary performance tests, in particular, forced expiration, is accurately and consistently administered can be difficult for lay people. Consequently, ambulatory pulmonary performance testing results generally lack a sufficient degree of reliability for use in medical diagnosis and treatment. Implantable medical devices facilitate ambulatory in situ physiological testing and monitoring, but conventional applications of implantable medical device measurement failed to provide an adequate solution to ambulatory pulmonary performance testing.

The '226 patent describes assessing pulmonary performance through transthoracic impedance monitoring. Transthoracic impedance measures are directly collected through an implantable medical device. The transthoracic impedance measures are correlated to pulmonary functional measures relative to performance of at least one respiration cycle. The transthoracic impedance measures are grouped into at least one measures set corresponding to one of an inspiratory phase and an expiratory phase. The at least one transthoracic impedance measures set are evaluated to identify a respiratory pattern relative to the inspiratory phase or the expiratory phase to represent pulmonary performance.

One pulmonary condition is snoring. The term snoring generally refers to a rough or hoarse sound that arises from a person's mouth while sleeping. The problems caused by snoring are both social, affecting those who sleep with or near the person snoring, and medical, sometimes signaling a more profound problem known as sleep apnea. During waking hours, normal tension in the muscles of the mouth and pharynx maintains a smooth airway in which air flows quietly, but as an individual falls asleep, these muscles become deeply relaxed. This can cause narrowing of the pharyngeal airway, which in turn causes turbulent airflow. This turbulent airflow vibrates the soft parts of the pharyngeal passage, causing the phenomenon we know as snoring. In children, enlarged tonsils or adenoids that obstruct the pharyngeal passageway can cause snoring. In adults, the contributing factors generally include a lack of muscle tone in the muscles of the airway, the consumption of alcohol or drugs, which causes a deeper relaxation, and smoking, which irritates the mucus membranes of the upper airway causing swelling and increased mucus production. Anatomical features can also play a part, such as a short neck or receding jaw line. Depending on the degree of blockage, there can be simple snoring or a momentary, total blockage of the airflow, known as obstructive sleep apnea. Obstructive sleep apnea is a potentially very serious condition. The oxygen starvation it induces can cause the person to partially awaken in order that muscle tension can open the airway and get air into their lungs. Apnea patients may experience 30 to 300 obstructed events per night, and many spend as much as half their sleep time with blood oxygen levels below normal. During their obstructive episodes, the heart must pump harder to circulate the blood faster. This condition can cause excessive daytime sleepiness, irregular heartbeats, and after many years it leads to elevated blood pressure and heart enlargement. Persons with obstructive sleep apnea may spend little of their nighttime hours in the deep sleep stages that are essential for a good rest. Therefore, they awaken un-refreshed and are sleepy much of the day. They can even fall asleep while driving or performing other activities.

U.S. Pat. No. 7,331,349 prevents snoring and sleep apnea by advancing the mandible of an individual during sleep. Instead of using an intra-oral device that has the potential to cause movement of the teeth, an extra-oral device is used, having a rigid headpiece, mandibular cradles that press against the posterior angle of the mandible, and a connector between the headpiece and the jaw pads to cause the force that maintains the mandible in the forward position to be transmitted to the head, rather than the teeth.

Bruxism has generally been defined as nonfunctional clenching, grinding, gritting, gnashing, and/or clicking of the teeth. Bruxism may occur while a person is awake or asleep. When the phenomenon occurs during sleep, it is called nocturnal bruxism. Even when it occurs during waking hours, the bruxer is often not conscious of the activity. Biting force exerted during bruxism often significantly exceeds peak biting force exerted during normal chewing. Chronic bruxism may result in musculoskeletal pain, headaches, and damage to the teeth and/or the temporomandibular joint Bruxism has been connected with temporomandibular disorders (TMD) or temporomandibular joint (TMJ) syndrome. U.S. Pat. No. 6,638,241 discloses an apparatus for the treatment of bruxism, including a biosensor adapted to sense a phenomenon associated with a bruxing event, and a relaxation stimulator in communication with the biosensor and adapted to provide a relaxation stimulus to relax at least one of an obruxism muscle and an obruxism nerve.

### SUMMARY

Systems and methods are disclosed for determining a pulmonary function by mounting one or more sensors intra-orally; capturing intra-oral data; and determining the pulmonary function based on an analysis of the intra-oral data.

Implementations of the above methods may include one or more of the following. The method determines an intermittent breathing condition from the intra-oral sound or determining a snoring condition from the intra-oral sound. The sensors are positioned in a custom removable appliance and the appliance can be secured to a tooth or a mandible using one of a screw, an adhesive, a fastener. The method can include measuring a magnitude and a frequency of an intra-oral sound; and determining one or more intervals between breaths from the intra-oral sound. The method includes capturing oxygen concentration, measuring carbon dioxide saturation, measuring oxygen data through a lax stratum corneum or a dermal structure. The sensors can perform a dual-color ratiometric oxygen saturation measurement. The sensors can also detect breath oxygen or carbon dioxide content. Inhaled and exhaled air can be measured for oxygen and/or carbon dioxide content. The system can provide a stimulus signal to a patient based on the pulmonary function, and the stimulus signal can be applied to a jaw. A sensation of sound, vibration or electrical stimulation can be generated. The method can cause the altering a depth of sleep through the stimulus signal. The system can alter a body position through the stimulus signal. The system can measure cardiac signals, EKG signals or ECG signals. An alarm can be generated based on the cardiac signals. The system can release a drug from an appliance. Intra-oral sensors can be mounted to a custom appliance. The sensors can be temperature sensors, flow velocity sensors, acoustic sensors, heart rate sensors, optical sensors, arterial tone sensors, oxygen sensors, EEG sensors, EKG sensors, pH sensors, or snoring sound sensors. The system can detect a sleep apnea condition, a snoring condition, a pulmonary condition, or a bruxing condition. The system can treat a sleep apnea condition, a snoring condition, a pulmonary condition, or a bruxing condition. The system can provide therapy to a patient. A vibration can be delivered to a tooth or a gum. The system can wake a patient. This can be done by delivering sound to wake a patient. The system can deliver electrical energy to stimulate nerves.

In another aspect, an apparatus for transmitting vibrations via at least one tooth to facilitate communications with a housing having a shape which is conformable to at least a portion of the at least one tooth; an actuatable transducer disposed within or upon the housing and in vibrator communication with a surface of the at least one tooth; and a pulmonary detector coupled to the transducer.

Implementations of the above aspect may include one or more of the following. The housing can be an oral appliance having a shape which conforms to the at least one tooth. The housing can be a custom removable appliance and wherein the housing, is secured to a tooth or a mandible using one of: a screw, an adhesive, a fastener.

The system provides a pulmonary monitoring means which is retained on the individual and thus is less subject to destruction, loss, forgetfulness, or any of the numerous other problems. The information helps the patient, treating professionals, and any other stakeholders to assist the patient in properly using, the appliance in a timely manner. The information can be displayed as a number, or can be displayed relative to an expected number that clinician specifies can be used in a display to provide feedback information.

### BRIEF DESCRIPTION OF THE DRAWINGS

FIG. 1 illustrates the dentition of a patient's teeth and one variation of a two-way communication device which is removably placed upon or against the patient's tooth or teeth as a removable oral appliance.

FIG. 1A shows an exemplary electronic system for assessing pulmonary function based on processing of intra-oral sound,

FIG. 1B shows a first exemplary process for assessing pulmonary function based on processing of intra-oral sound.

FIG. 1C shows a second exemplary process for assessing pulmonary function based on processing of intra-oral sound.

FIG. 1D shows a third exemplary process for assessing pulmonary function based on processing of intra-oral sound.

FIG. 1E shows another exemplary process for processing the intra-oral sound.

FIG. 2A illustrates a perspective view of the lower teeth showing one exemplary location for placement of the removable oral appliance two-way communication device.

FIG. 2B illustrates another variation of the removable oral appliance in the form of an appliance which is placed over an entire row of teeth in the manner of a mouthguard.

FIG. 2C illustrates another variation of the removable oral appliance which is supported by an arch.

FIG. 2D illustrates another variation of an oral appliance configured as a mouthguard.

FIG. 3 illustrates a detail perspective view of the oral appliance positioned upon the patient's teeth utilizable in combination with a transmitting assembly external to the mouth and wearable by the patient in another variation of the device.

FIG. 4 shows an illustrative configuration of the individual components in a variation of the oral appliance device having an external transmitting assembly with a receiving and transducer assembly within the mouth.

FIG. 5 shows an illustrative configuration of another variation of the device in which the entire assembly is contained by the oral appliance within the user's mouth.

FIG. 6A shows a partial cross-sectional view of an oral appliance placed upon a tooth with an electronics/transducer assembly adhered to the tooth surface via an adhesive.

FIG. 6B shows a partial cross-sectional view of a removable backing adhered onto an adhesive surface.

FIG. 7 shows a partial cross-sectional view of another variation of an oral appliance placed upon a tooth with an electronics/transducer assembly pressed against the tooth surface via an osmotic pouch.

FIG. 8 shows a partial cross-sectional view of another variation of an oral appliance placed upon a tooth with an electronics/transducer assembly pressed against the tooth surface via one or more biasing elements.

FIG. 9 illustrates another variation of an oral appliance having an electronics assembly and a transducer assembly separated from one another within the electronics and transducer housing of the oral appliance.

FIGS. 10 and 11 illustrate additional variations of oral appliances in which the electronics and transducer assembly are maintainable against the tooth surface via a ramped surface and a biasing element.

FIG. 12 shows yet another variation of an oral appliance having an interfacing member positioned between the electronics and/or transducer assembly and the tooth surface.

FIG. 13 shows yet another variation of an oral appliance having an actuatable mechanism for urging the electronics and/or transducer assembly against the tooth surface.

FIG. 14 shows yet another variation of an oral appliance having a cam mechanism for urging the electronics and/or transducer assembly against the tooth surface.

FIG. 15 shows yet another variation of an oral appliance having a separate transducer mechanism positionable upon the occlusal surface of the tooth for transmitting vibrations.

FIG. 16 illustrates another variation of an oral appliance having a mechanism for urging the electronics and/or transducer assembly against the tooth surface utilizing a bite-actuated mechanism.

FIG. 17 shows yet another variation of an oral appliance having a composite dental anchor for coupling the transducer to the tooth.

FIGS. 18A and 18B show side and top views, respectively, of an oral appliance variation having one or more transducers which may be positioned over the occlusal surface of the tooth.

FIGS. 19A and 198 illustrate yet another variation of an oral appliance made from a shape memory material in its pre-formed relaxed configuration and its deformed configuration when placed over or upon the patient's tooth, respectively, to create an interference fit.

FIG. 20 illustrates yet another variation of an oral appliance made from a pre-formed material in which the transducer may be positioned between the biased side of the oral appliance and the tooth surface.

FIG. 21 illustrates a variation in which the oral appliance may be omitted and the electronics and/or transducer assembly may be attached to a composite dental anchor attached directly to the tooth surface.

FIGS. 22A and 22B show partial cross-sectional side and perspective views, respectively, of another variation of an oral appliance assembly having its occlusal surface removed or omitted for patient comfort.

FIGS. 23A and 238 illustrate perspective and side views, respectively, of an oral appliance which may be coupled to a screw or post implanted directly into the underlying bone, such as the maxillary or mandibular bone.

FIG. 24 illustrates another variation in which the oral appliance may be coupled to a screw or post implanted directly into the palate of a patient.

FIGS. 25A and 25B illustrate perspective and side views, respectively, of an oral appliance which may have its transducer assembly or a coupling member attached to the gingival surface to conduct vibrations through the gingival tissue and underlying bone.

FIG. 26 illustrates an example of how multiple oral appliance two-way communication assemblies or transducers may he placed on multiple teeth throughout the patient's mouth.

FIGS. 27A and 27B illustrate perspective and side views, respectively, of an oral appliance (similar to a variation shown above) which may have a microphone unit positioned adjacent to or upon the gingival surface to physically separate the microphone from the transducer to attenuate or eliminate feedback.

FIG. 28 illustrates another variation of a removable oral appliance supported by an arch and having a microphone unit integrated within the arch.

FIG. 29 shows yet another variation illustrating at least one microphone and optionally additional microphone units positioned around the user's mouth and in wireless communication with the electronics and/or transducer assembly.

### DESCRIPTION

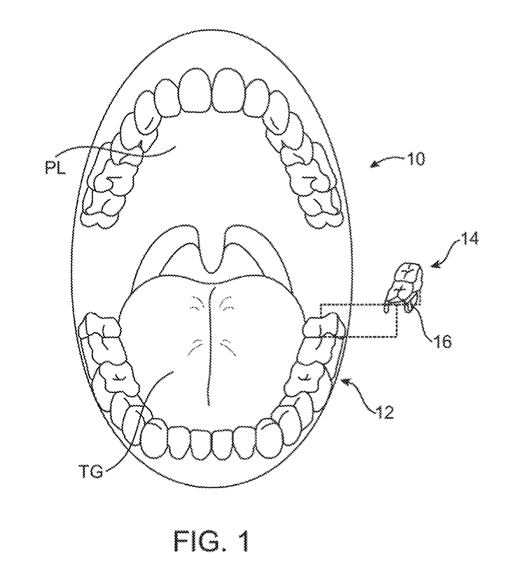

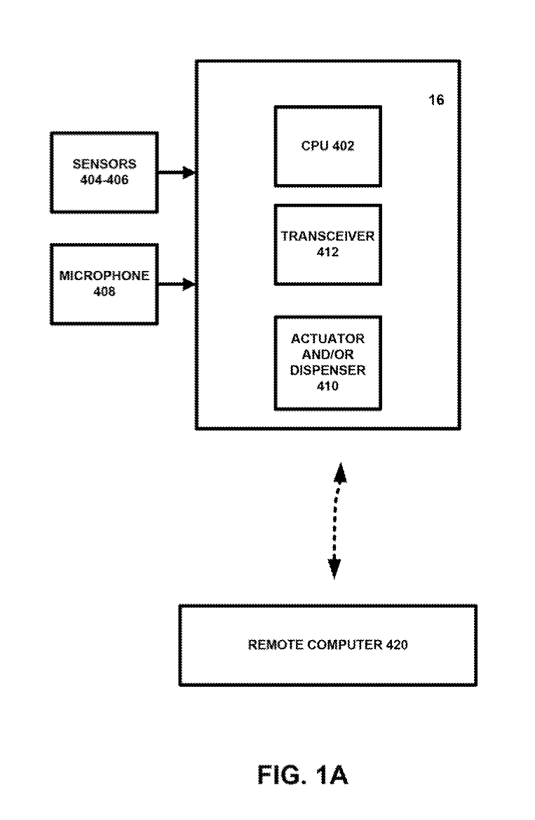

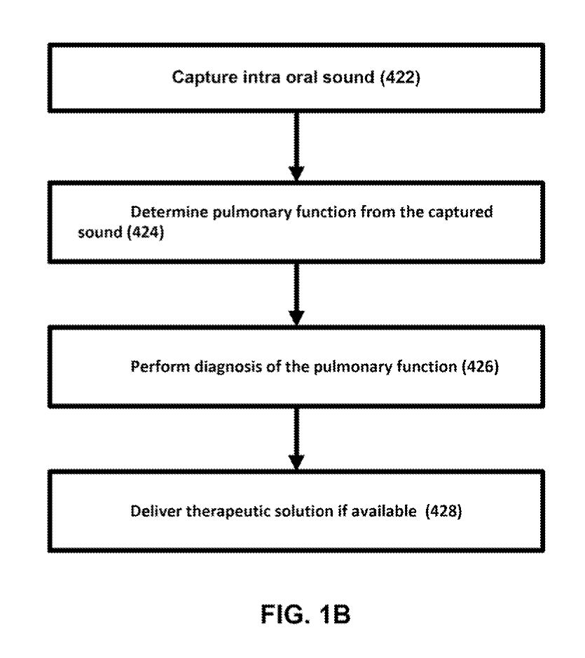

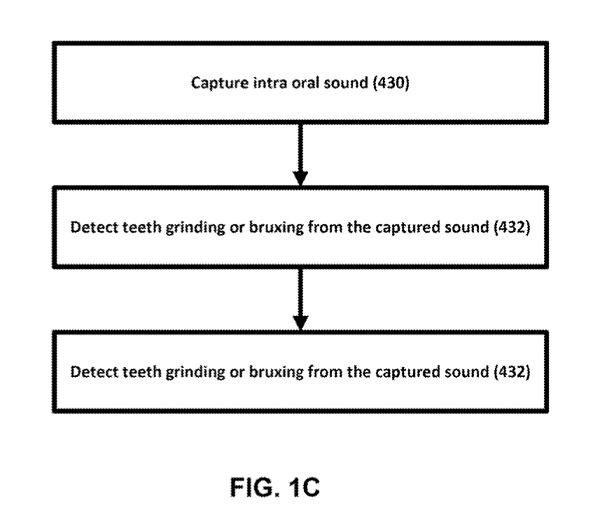

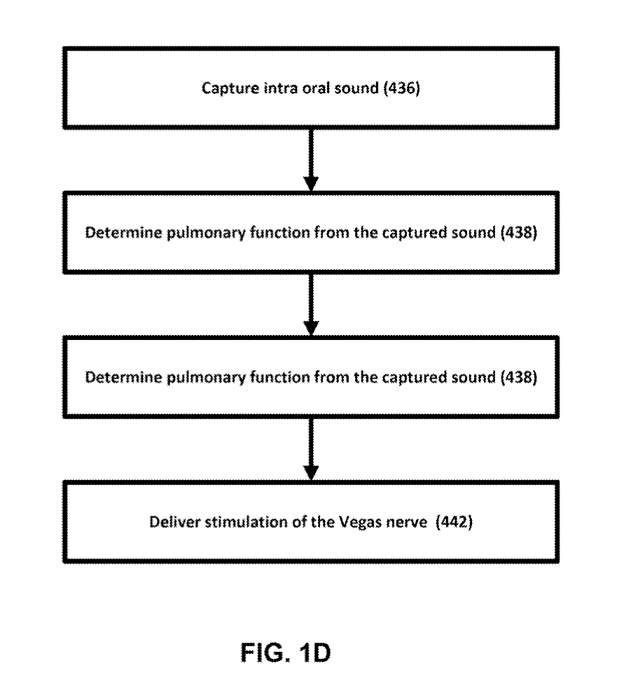

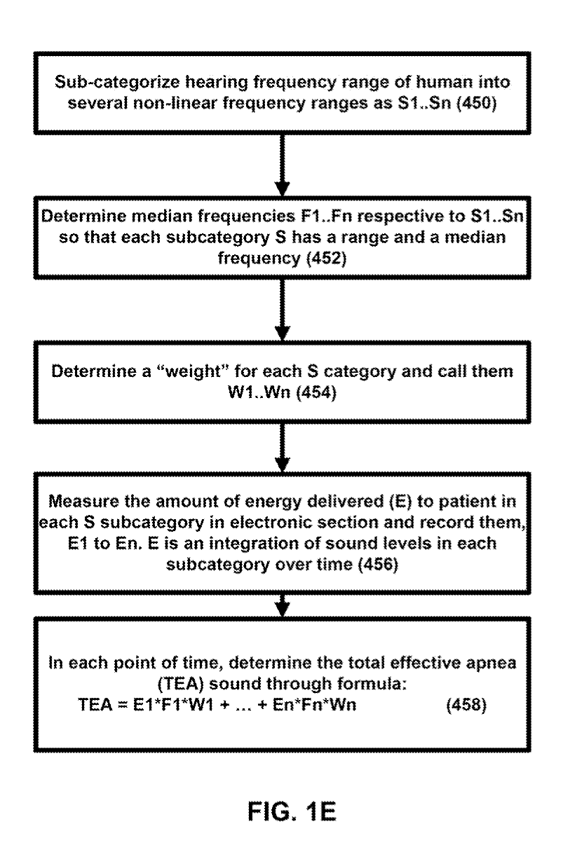

FIG. 1 shows an exemplary intra-oral pulmonary monitoring and/or treatment device or appliance 1. In one embodiment, the device or appliance 1 is positioned next to the upper molars of the human teeth. In one embodiment, the device or appliance 1 receives microphone signals from inside or outside the body and converting them to vibrations that can be transmitted by the upper molar through the skull to the eardrums. As the apparatus of FIG. 1 is intraoral, it can perform sleep apnea monitoring at night in a non-invasive and comfortable environment for the patient.

In one embodiment, the device or appliance 1 performs intraoral sound monitoring. The device or appliance or apparatus can be used to measure sound volume and frequencies that are associated with sleep apnea, for example snoring sounds, intermittent breathing sounds, and intervals between breaths. Sleep studies are normally performed in sleep labs by appointment and only periodically, as they are an involved process and are inconvenient for the patient to attend. The intraoral apparatus can monitor sleep apnea as frequently as necessary and possibly every night, if the patient is already fitted with one as a hearing aid.

In another embodiment, the device or appliance or apparatus 1 can monitor oxygen saturation. The proximity of the aforementioned apparatus to the gum tissue provides a location for oxygen saturation monitoring through lax stratum corneum and other dermal structures. The oxygen saturation information can be captured using dual-color ratiometric oxygen saturation measurements, for example.

In yet another embodiment, the system can monitor oxygen and carbon dioxide contents and inhaled and exhaled by the patient. As the inhaled, and exhaled air passes by, the apparatus can measure the inhaled and exhaled air for oxygen and carbon dioxide content to provide additional diagnostic information in terms of the amount of oxygen that is extracted from the inhaled air.

The system can also provide stimulations to the patient in another embodiment. By applying a stimulation signal to the jaw, one can alter the depth of sleep, as well as potentially body position by the response of the subject to a mild tingling sensation.

In another embodiment, the system can perform EKG monitoring. The EKG signals can be picked up through the intraoral tissues, and the EKG signal can provide additional indication of dangerous condition(s) that may arise while a person is sleeping. An external alarm can then be triggered to wake the person or their caregiver to alert them of such a condition.

In addition to handling pulmonary functions, the device or appliance of FIG. 1 can handle other functions such as communication and patient identification functions, among others. For example, in one embodiment, the treatment device or appliance provides an electronic and transducer device that can be attached, adhered, or otherwise embedded into or upon a removable oral appliance or other oral device to form a two-way communication assembly. In another embodiment, the device 1 provides an electronic and transducer device that can be attached, adhered, or otherwise embedded into or upon a removable oral appliance or other oral device to form a medical tag containing patient identifiable information. Such an oral appliance may be a custom-made device fabricated from a thermal forming process utilizing a replicate model of a dental structure obtained, by conventional dental impression methods. The electronic and transducer assembly may receive incoming sounds either directly or through a receiver to process and amplify the signals and transmit the processed sounds via a vibrating transducer element coupled to a tooth or other bone structure, such as the maxillary, mandibular, or palatine bone structure.

Turning now to more details on the device or appliance 1 as shown in FIG. 1, a patient's mouth and dentition 10 is illustrated showing one possible location for removably attaching a pulmonary assessment device or assembly 14 upon or against at least one tooth, such as a molar 12. The patient's tongue TG and palate PL are also illustrated for reference. An electronics and/or transducer assembly 16 may be attached, adhered, or otherwise embedded into or upon the assembly 14, as described below in further detail.

FIG. 1A shows in more details the assembly 16. In this embodiment, a central processing unit (CPU) 402 communicates with one or more sensors 404-406. The CPU 402 also captures sound through a microphone 408. The CPU 402 can cause an actuator 410 to vibrate or to deliver medication, for example. The CPU 402 can also transmit data to a remote computer 420 using, a transceiver 412.

The custom appliance 14/16 can perform diagnostic and therapy delivery for sleep apnea, snoring, pulmonary, teeth grinding, among others. Built-in sensors 404-406 such as temperature sensors, flow velocity sensors, acoustic sensors, heart rate sensors, optical sensors, arterial tone sensors, oxygen sensors, and various electrical sensors such as EEG sensor, EKG sensor, pH sensor, and snoring sound sensor can be deployed.

The temperature sensors can be infrared (IR) thermometers, thermal imagers, RTDs & PRTs, thermistors, thermocouples, or thermometers. The flow velocity sensors can be micro-electromechanical systems (MEMs) devices. The acoustic sensors can he microphones or MEMS sensor. The heart rate sensors can electronically sense the human heartbeat and can be done acoustically (stethoscope or Doppler), mechanically (sphygmomanometer), electrically (EKG), and optically. One optical technique exploits the fact that tiny subcutaneous blood vessels (capillaries) in any patch of skin (fingertip, ear lobe, etc.) furnished with a good blood supply, alternately expand and contract in time with the heartbeat. Alternatively a piezoelectric sensor can measure the heart rate by detecting the micro movements of the body associated to the ejection of blood in the aorta and the output signal is amplified and filtered to serve in further signal processing. Other heart rate sensing techniques known to one skilled in the art can be used as well.

The EKG or ECG (electrocardiogram) is a test that measures the electrical activity of the heartbeat. With each beat, an electrical impulse (or “wave”) travels through the heart. This wave causes the muscle to squeeze and pump blood from the heart A normal heartbeat on ECG will show the timing, of the top and lower chambers. The right and left atria or upper chambers make the first wave called a “P wave”—following a flat line when the electrical impulse goes to the bottom chambers. The right and left bottom chambers or ventricles make the next wave called a “QRS complex.” The final wave or “T wave” represents electrical recovery or return to a resting, state for the ventricles. An ECG gives two major kinds of information. First, by measuring time intervals on the ECG, a doctor can determine how long the electrical wave takes to pass through the heart. Finding out how long a wave takes to travel from one part of the heart to the next shows if the electrical activity is normal or slow, fast or irregular. Second, by measuring the amount of electrical activity passing through the heart muscle, a cardiologist may be able to find out if parts of the heart are too large or are overworked.

The pH sensor measures the acidity or alkalinity of a solution. Aqueous solutions at 25° C. with a pH less than 7 are considered acidic, while those with a pH greater than 7 are considered basic (alkaline). pH values in water are commonly in the range 0-14, though more extreme values, even negative values, are possible. When a pH level is 7.0, it is defined as ‘neutral’ at 25° C. because at this pH the concentration of H₃O⁺ equals the concentration of H⁻ in pure water.

The actuator 410 provides therapy when pulmonary conditions warrant. The actuator 410 can be an electrical energy source to provide shock. The actuator can be a sound source such as a speaker to provide sound. The actuator 410 can be a buzzer or a vibrator to provide vibration. The actuator 410 can also be an electrically actuated drug reservoir that provides drug release when conditions warrant such release. Exemplary conditions that can be monitored and/or treated by the appliance include sleep apnea and pulmonary monitoring, teeth grinding/bruxing, and stimulation of Vegas nerve, among others. The system of FIG. 1A can provide diagnosis and therapy delivery via custom made appliance for sleep apnea, snoring, pulmonary function and for bruxing. The sensors can include sensors for oxygen and carbon dioxide saturation, Temperature, Air Flow Velocity, acoustic sound, heart rate, arterial tone, Electrical (EEG & EKG, PH), respiratory cycle, among others. FIGS. 1B-1D show exemplary processes that allow the unit can provide therapy through a delivery of vibration on the tooth or gum to wake the patient, a delivery of sound to wake the patient, or a delivery of electrical energy to stimulate the nerves.

Turning now to FIG. 1B, a first exemplary process for assessing pulmonary function based on processing of intra-oral sound is shown. In this process, the CPU 402 captures intra oral sound (422) and determines pulmonary function from the captured sound (424). Next, the CPU 402 performs diagnosis of the pulmonary function (426). The system delivers therapeutic solution if available (428).

FIG. 1C shows a second exemplary process for assessing pulmonary function based on processing of intra-oral sound. The process includes capturing intra oral sound (430), detecting teeth grinding or bruxing from the captured sound (432), and delivering vibration on teeth or gum to wake up patient (434).

Referring now to FIG. 1D, a third exemplary process is shown for assessing pulmonary function based on processing of intra-oral sound. In this process, the processor captures intra oral sound (436) and determines pulmonary function from the captured sound (438). Next, the system performs diagnosis of the pulmonary function (440) and delivers a stimulation of the Vegas nerve if needed (442).

FIG. 1E shows another exemplary process for processing the intra-oral sound. In this embodiment, the system sub-categorizes hearing frequency range of human into several non-linear frequency ranges S1 . . . Sn (450). In one embodiment, n is between five and eight. Each subcategory S has a range and a median frequency, and the system determines median frequencies F1 . . . Fn respectively for S1 . . . Sn (452). Next, the system specifies a “weight” for each S category as W1 to Wn (454), Weights are by the default 1 and the weight may change them later based on clinical trials. The system then measures the amount of energy delivered (E) to patient in each S subcategory in electronic section and record them. E1 to En (456). E is an integration of sound levels in each subcategory over time. For each point of time the system calculates the total effective apnea sound to the patient through the formula:

TEA=E1*F1*W1+ . . . En*Fn*Wn(458)

TEA can be accumulated over time as an indicator for patient's apnea condition or relative to an expected number that clinician specifies can be used in a Patient Control Unit display. In case of relative number it can be a 0 to 100% for ease of understanding. The pseudo-code is as follows:

Sub-categorize hearing frequency range of human into several non-linear frequency ranges as S1 . . . Sn (450).

Determine median frequencies F1 . . . Fn respective to S1 . . . Sn so that each subcategory S has a range and a median frequency (452)

Determine a “weight” for each S category and call them W1 . . . Wn (454).

Measure the amount of energy delivered (E) to patient in each S subcategory in electronic section and record them, E1 to En. E is an integration of sound levels in each subcategory over time (456)

In each point of time, determine the total effective apnea (TEA) sound through formula:

TEA=E1*F1*W1+ . . . En*Fn*Wn(458)

TEA can be scaled as a relative number between 0 and 100 to provide an expected number for each patient and can be adjusted to be between the 0 to 100 range (Relative TEA). Such relative TEA scaled number provides an indicator of patient exposure to the sound delivered by the system.

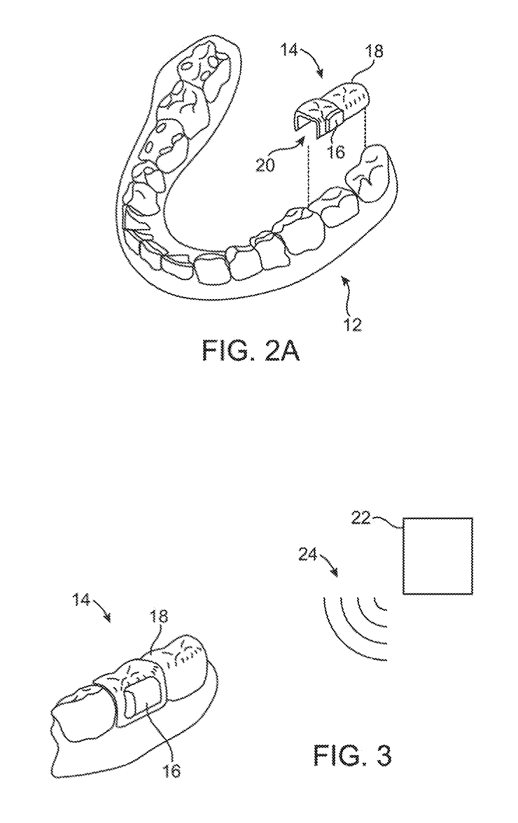

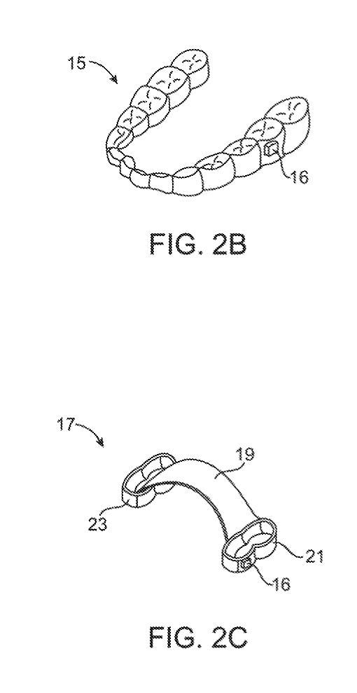

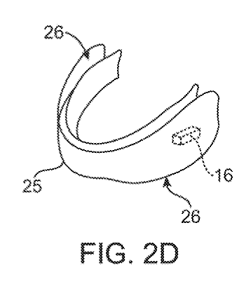

FIG. 2A shows a perspective view of the patient's lower dentition illustrating the two-way communication assembly 14 comprising, a removable oral appliance 18 and the electronics and/or transducer assembly 16 positioned along a side surface of the assembly 14. In this variation, oral appliance 18 may be fitted upon two molars 12 within tooth engaging channel 20 defined by oral appliance 18 for stability upon the patient's teeth, although in other variations, a single molar or tooth may be utilized. Alternatively, more than two molars may be utilized, for the oral appliance 18 to be attached upon or over. Moreover, electronics and/or transducer assembly 16 is shown positioned upon a side surface of oral appliance 18 such that the assembly 16 is aligned along a buccal surface of the tooth 12; however, other surfaces such as the lingual surface of the tooth 12 and other positions may also be utilized. The figures are illustrative of variations and are not intended to be limiting; accordingly, other configurations and shapes for oral appliance 18 are intended to be included herein.

FIG. 2B shows another variation of a removable oral appliance in the form of an appliance 15 which is placed over an entire row of teeth in the manner of a mouthguard. In this variation, appliance 15 may be configured to cover an entire bottom row of teeth or alternatively an entire upper row of teeth. In additional variations, rather than covering the entire rows of teeth, a majority of the row of teeth may be instead be covered by appliance 15. Assembly 16 may be positioned along one or more portions of the oral appliance 15.

FIG. 2C shows yet another variation of an oral appliance 17 having an arched configuration. In this appliance, one or more tooth retaining portions 21, 23, which in this variation may be placed along the upper row of teeth, may be supported by an arch 19 which may lie adjacent or along the palate of the user. As shown, electronics and/or transducer assembly 16 may be positioned along one or more portions of the tooth retaining portions 21, 23. Moreover, although the variation shown illustrates an arch 19 which may cover only a portion of the palate of the user, other variations may be configured to have an arch which covers the entire palate of the user.

FIG. 2D illustrates yet another variation of an oral appliance in the form of a mouthguard or retainer 25 which may be inserted and removed easily from the user's mouth. Such a mouthguard or retainer 25 may be used in sports where conventional mouthguards are worn; however, mouthguard or retainer 25 having assembly 16 integrated therein may be utilized by persons, hearing impaired or otherwise, who may simply hold the mouthguard or retainer 25 via grooves or channels 26 between their teeth for receiving instructions remotely and communicating over a distance.

Generally, the volume of electronics and/or transducer assembly 16 may be minimized, so as to be unobtrusive and as comfortable to the user when placed in the mouth. Although the size may be varied, a volume of assembly 16 may be less than 800 cubic millimeters. This volume is, of course, illustrative and not limiting as size and volume of assembly 16 and may be varied accordingly between different users.

Moreover, removable oral appliance 18 may be fabricated from various polymeric or a combination of polymeric and metallic materials using any number of methods, such as computer-aided machining processes using computer numerical control (CNC) systems or three-dimensional printing processes, e.g., stereolithography apparatus (SLA), selective laser sintering (SLS), and/or other similar processes utilizing three-dimensional geometry of the patient's dentition, which may be obtained via any number of techniques. Such techniques may include use of scanned dentition using intra-oral scanners such as laser, white light, ultrasound, mechanical three-dimensional touch scanners, magnetic resonance imaging (MRI), computed tomography (CT), other optical methods, etc.

In forming the removable oral appliance 18, the appliance 18 may be optionally formed such that it is molded to fit over the dentition and at least a portion of the adjacent gingival tissue to inhibit the entry of food, fluids, and other debris into the oral appliance 18 and between the transducer assembly and tooth surface. Moreover, the greater surface area of the oral appliance 18 may facilitate the placement and configuration of the assembly 16 onto the appliance 18.

Additionally, the removable oral appliance 18 may be optionally fabricated to have a shrinkage factor such that when placed onto the dentition, oral appliance 18 may be configured to securely grab onto the tooth or teeth as the appliance 18 may have a resulting size slightly smaller than the scanned tooth or teeth upon which the appliance 18 was formed. The fitting may result in a secure interference fit between the appliance 18 and underlying dentition.

In one variation, with assembly 14 positioned upon the teeth, as shown in FIG. 3, an extra-buccal transmitter assembly 22 located outside the patient's mouth may be utilized to receive auditory signals for processing and transmission via a wireless signal 24 to the electronics and/or transducer assembly 16 positioned within the patient's mouth, which may then process and transmit the processed auditory signals via vibratory conductance to the underlying tooth and consequently to the patient's inner ear.

The transmitter assembly 22, as described in further detail below, may contain a microphone assembly as well as a transmitter assembly and may be configured in any number of shapes and forms worn by the user, such as a watch, necklace, lapel, phone, belt-mounted device, etc.

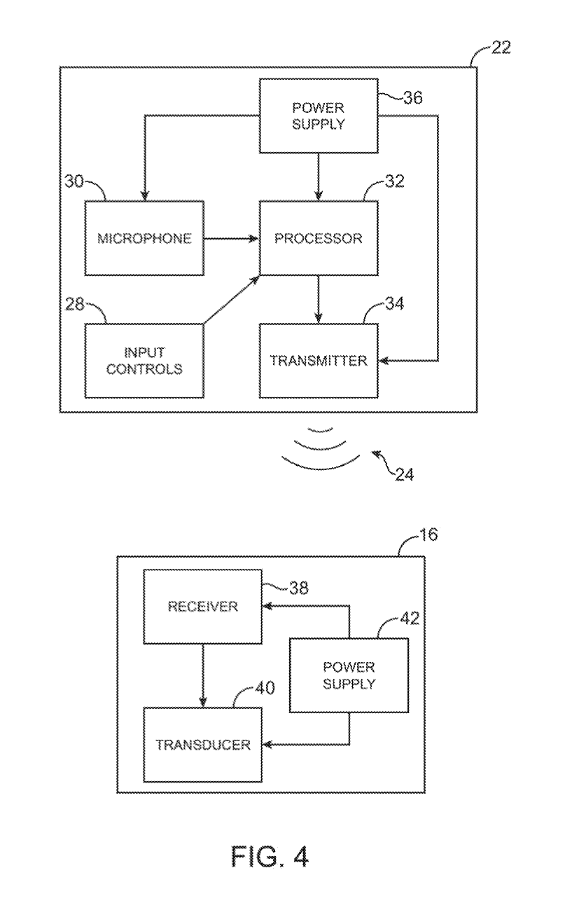

FIG. 4 illustrates a schematic representation of one variation of two-way communication assembly 14 utilizing an extra-buccal transmitter assembly 22, which may generally comprise microphone 30 for receiving sounds and which is electrically connected to processor 32 for processing the auditory signals. Processor 32 may be connected electrically to transmitter 34 for transmitting the processed signals to the electronics and/or transducer assembly 16 disposed upon or adjacent to the user's teeth. The microphone 30 and processor 32 may be configured to detect and process auditory signals in any practicable range, but may be configured in one variation to detect auditory signals ranging from, e.g., 250 Hertz to 20,000 Hertz.

With respect to microphone 30, a variety of various microphone systems may be utilized. For instance, microphone 30 may be a digital, analog, and/or directional type microphone. Such various types of microphones may be interchangeably configured to be utilized with the assembly, if so desired.

Power supply 36 may be connected to each of the components in transmitter assembly 22 to provide power thereto. The transmitter signals 24 may be in any wireless form utilizing, e.g., radio frequency, ultrasound, microwave, Blue Tooth® (BLUETOOTH SIG, INC., Bellevue, Wash.), etc. for transmission to assembly 16. Assembly 22 may also optionally include one or more input controls 28 that a user may manipulate to adjust various acoustic parameters of the electronics and/or transducer assembly 16, such as acoustic focusing, volume control, filtration, muting, frequency optimization, sound adjustments, and tone adjustments, etc.

The signals transmitted 24 by transmitter 34 may be received by electronics and/or transducer assembly 16 via receiver 38, which may be connected to an internal processor for additional processing of the received signals. The received signals may be communicated to transducer 40, which may vibrate correspondingly against a surface of the tooth to conduct the vibratory signals through the tooth and bone and subsequently to the middle ear to facilitate hearing of the user. Transducer 40 may be configured as any number of different vibratory mechanisms. For instance, in one variation, transducer 40 may be an electromagnetically actuated transducer. In other variations, transducer 40 may be in the form of a piezoelectric crystal having a range of vibratory frequencies, e.g., between 250 to 4000 Hz.

Power supply 42 may also be included with assembly 16 to provide power to the receiver, transducer, and/or processor, if also included. Although power supply 42 may be a simple battery, replaceable or permanent, other variations may include a power supply 42 which is charged by inductance via an external charger. Additionally, power supply 42 may alternatively be charged via direct coupling to an alternating current (AC) or direct current (DC) source. Other variations may include a power supply 42 which is charged via a mechanical mechanism, such as an internal pendulum or slidable electrical inductance charger as known in the art, which is actuated via, e.g., motions of the jaw and/or movement for translating, the mechanical motion into stored electrical energy for charging power supply 42.

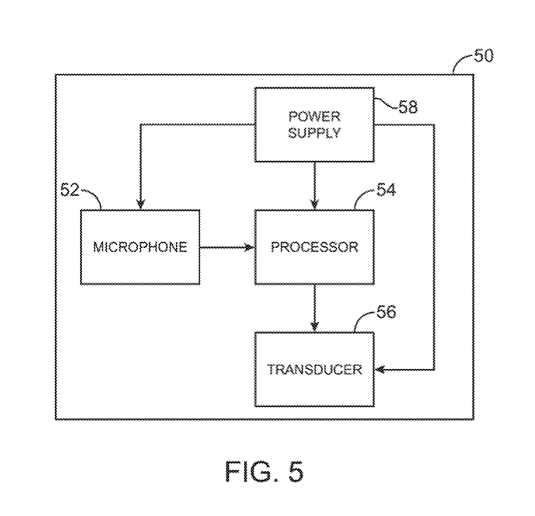

In another variation of assembly 16, rather than utilizing an extra-buccal transmitter, two-way communication assembly 50 may be configured as an independent assembly contained entirely within the user's mouth, as shown in FIG. 5. Accordingly, assembly 50 may include an internal microphone 52 in communication with an on-board processor 54. Internal microphone 52 may comprise any number of different types of microphones, as described above. Processor 54 may be used to process any received auditory signals for filtering and/or amplifying the signals and transmitting them to transducer 56, which is in vibratory contact against the tooth surface. Power supply 58, as described above, may also be included within assembly 50 for providing power to each of the components of assembly 50 as necessary.

In order to transmit the vibrations corresponding to the received auditory signals efficiently and with minimal loss to the tooth or teeth, secure mechanical contact between the transducer and the tooth is ideally maintained to ensure efficient vibratory communication. Accordingly, any number of mechanisms may be utilized to maintain this vibratory communication.

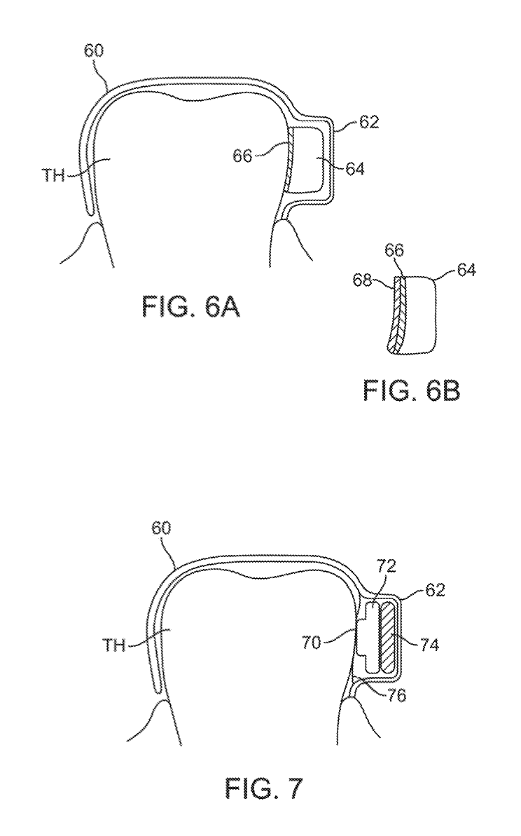

In one variation as shown in FIG. 6A, a partial cross-sectional view of a removable oral appliance 60 is shown placed over or upon a tooth TH. Electronics and/or transducer housing 62 may be seen defined along oral appliance 60 such that housing 62 is aligned or positioned adjacent to a side surface, buccal and/or lingual surface, of the tooth TH. Housing 62 may provide protection to the electronics and/or transducer assembly from the environment of the mouth.

An electronics and/or transducer assembly 64 may be simply placed, embedded, or encapsulated within housing 62 for contacting the tooth surface. In this variation, assembly 64 may be adhered against the tooth surface via an adhesive surface or film 66 such that contact is maintained between the two. As shown in FIG. 6B, a removable backing 68 may be adhered onto adhesive surface 66 and removed prior to placement upon the tooth surface. In this manner, assembly 64 may be replaced upon the tooth as necessary with additional electronics and/or transducer assemblies.

Aside from an adhesive film 66, another alternative may utilize an expandable or swellable member to ensure a secure mechanical contact of the transducer against the tooth. As shown in FIG. 7, an osmotic patch or expandable hydrogel 74 may be placed between housing 62 and electronics and/or transducer assembly 72. After placement of oral appliance 60, hydrogel 74 may absorb some fluids, either from any surrounding fluid or from a fluid introduced into hydrogel 74, such that hydrogel 74 expands in size to force assembly 72 into contact against the tooth surface. Assembly 72 may be configured to define a contact surface 70 having a relatively smaller contact area to facilitate uniform contact of the surface 70 against the tooth. Such a contact surface 70 may be included in any of the variations described herein. Additionally, a thin encapsulating layer or surface 76 may be placed over housing 62 between contact surface 70 and the underlying tooth to prevent any debris or additional fluids from entering housing 62.

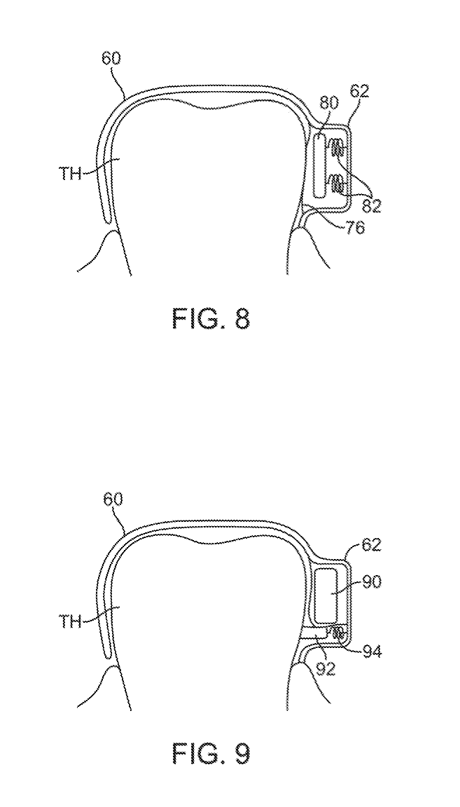

Another variation is shown in FIG. 8, which shows electronics and/or transducer assembly 80 contained within housing 62. In this variation, one or more biasing elements 82, e.g., springs, pre-formed shape memory elements, etc., may be placed between assembly 80 and housing 62 to provide a pressing force on assembly 80 to urge the device against the underlying tooth surface, thereby ensuring mechanical contact.

In yet another variation, the electronics may be contained as a separate assembly 90 which is encapsulated within housing 62 and the transducer 92 may be maintained separately from assembly 90 but also within housing 62. As shown in FIG. 9, transducer 92 may be urged against the tooth surface via a spring or other biasing element 94 and actuated via any of the mechanisms described above.

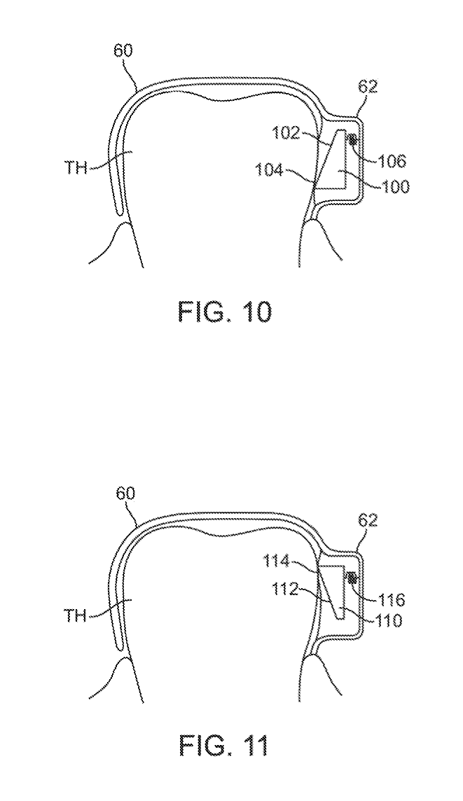

In other variations as shown in FIG. 10, electronics and/or transducer assembly 100 may be configured to have a ramped surface 102 in apposition to the tooth surface. The surface 102 may be angled away from the occlusal surface of the tooth. The assembly 100 may be urged via as biasing element or spring 106 which forces the ramped surface 102 to pivot about a location 104 into contact against the tooth to ensure contact for the transducer against the tooth surface.

FIG. 11 illustrates another similar variation in electronics and/or transducer assembly 110 also having a ramped surface 112 in apposition to the tooth surface. In this variation, the ramped surface 112 may be angled towards the occlusal surface of the tooth. Likewise, assembly 110 may be urged via a biasing clement or spring 116 which urges the assembly 110 to pivot about its lower end such that the assembly 110 contacts the tooth surface at a region 114.

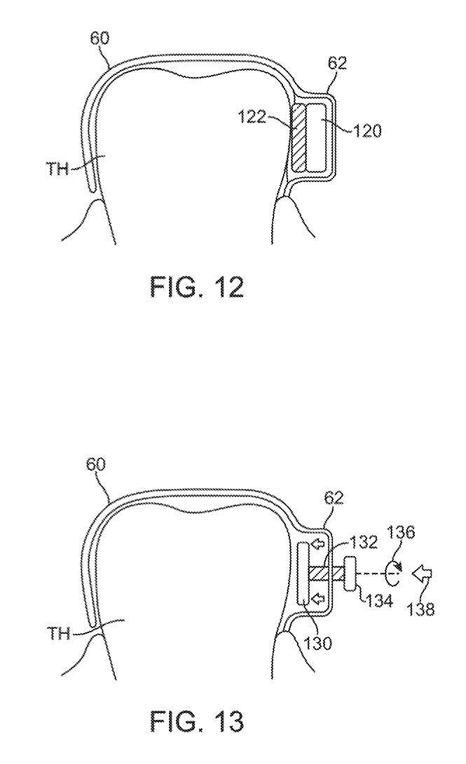

In yet another variation shown in FIG. 12, electronics and/or transducer assembly 120 may be positioned within housing 62 with an interface layer 122 positioned between the assembly 120 and the tooth surface. Interface layer 122 may be configured to conform against the tooth surface and against assembly 120 such that vibrations may be transmitted through layer 122 and to the tooth in a uniform manner. Accordingly, interface layer 122 may be made from a material which attenuates vibrations minimally. Interface layer 122 may be made in a variety of forms, such as a simple insert, an O-ring configuration, etc. or even in a gel or paste form, such as denture or oral paste, etc. Additionally, layer 122 may be fabricated from various materials, e.g., hard plastics or polymeric materials, metals, etc.

FIG. 13 illustrates yet another variation in which electronics and/or transducer assembly 130 may be urged against the tooth surface via a mechanical mechanism. As shown, assembly 130 may be attached to a structural member 132, e.g., a threaded member or a simple shaft, which is connected through housing 62 to an engagement member 134 located outside housing 62. The user may rotate engagement member 134 (as indicated by rotational arrow 136) or simply push upon member 134 (as indicated by linear arrow 138) to urge assembly 130 into contact against the tooth. Moreover, actuation of engagement member 134 may he accomplished manually within the mouth or through the user's cheek or even through manipulation via the user's tongue against engagement member 134.

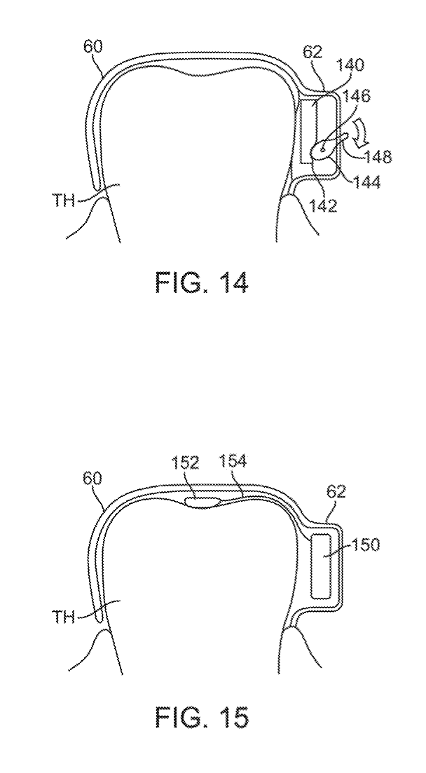

Another variation for a mechanical mechanism is illustrated in FIG. 14. In this variation, electronics and/or transducer assembly 140 may define a portion as an engaging surface 142 for contacting against a cam or lever mechanism 144. Cam or lever mechanism 144 may he configured to pivot 146 such that actuation of a lever 148 extending through housing 62 may urge cam or lever mechanism 144 to push against engaging surface 142 such that assembly 140 is pressed against the underlying tooth surface.

In yet another variation, the electronics 150 and the transducer 152 may be separated from one another such that electronics 150 remain disposed within housing 62 but transducer 152, connected via wire 154, is located beneath dental oral appliance 60 along an occlusal surface of the tooth, as shown in FIG. 15. In such a configuration, vibrations are transmitted via the transducer 152 through the occlusal surface of the tooth. Additionally, the user may bite down upon the oral appliance 60 and transducer 152 to mechanically compress the transducer 152 against the occlusal surface to further enhance the mechanical contact between the transducer 152 and underlying tooth to further facilitate transmission therethrough.

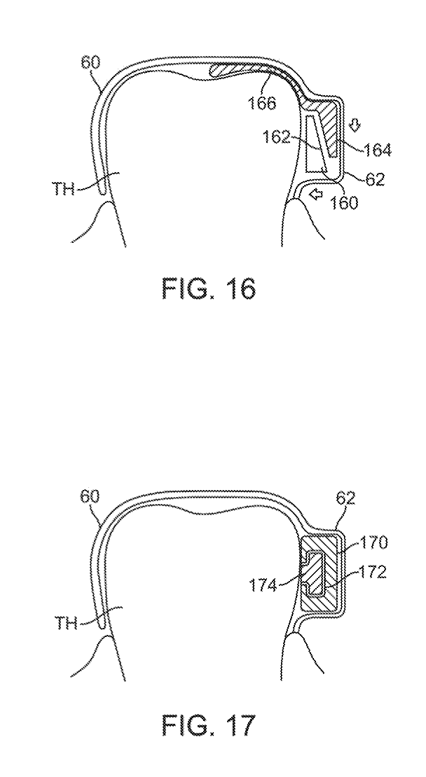

In the variation of FIG. 16, another example for a bite-enhanced coupling mechanism is illustrated where electronics and/or transducer assembly 160 defines an angled interface surface 162 in apposition to a correspondingly angled engaging member 164. A proximal end of engaging member 164 may extend through housing 62 and terminate in a pusher member 166 positioned over an occlusal surface of the tooth TH. Once oral appliance 60 is initially placed over tooth TH, the user may bite down or otherwise press down upon the top portion of oral appliance 60, thereby pressing down upon pusher member 166 which in turn pushes down upon engaging member 164, as indicated by the arrow. As engaging member 164 is urged downwardly towards the gums, its angled surface may push upon the corresponding and oppositely angled surface 162 to urge assembly 160 against the tooth surface and into a secure mechanical contact.

In yet another variation, an electronics and/or transducer assembly 170 may define a channel or groove 172 along a surface for engaging a corresponding dental anchor 174, as shown in FIG. 17. Dental anchor 174 may comprise a light-curable acrylate-based composite material adhered directly to the tooth surface. Moreover dental anchor 174 may be configured in a shape which corresponds to a shape of channel or groove 172 such that the two may be interfitted in a mating engagement. In this manner, the transducer in assembly 170 may vibrate directly against dental anchor 174 which may then transmit these signals directly into the tooth

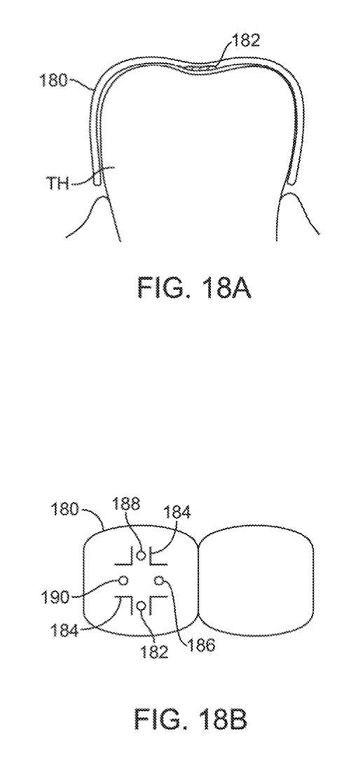

FIGS. 18A and 18B show partial cross-sectional side and top views, respectively, of another variation in which oral appliance 180 may define a number of channels or grooves 184 along a top portion of oral appliance 180. Within these channels or grooves 184, one or more transducers 182, 186, 188, 190 may be disposed such that the are in contact with the occlusal surface of the tooth and each of these transducers may be tuned to transmit frequencies uniformly. Alternatively, each of these transducers may be tuned to transmit only at specified frequency ranges. Accordingly, each transducer can be programmed or preset for a different frequency response such that each transducer may be optimized for a different frequency response and/or transmission to deliver a relatively high-fidelity sound to the user.

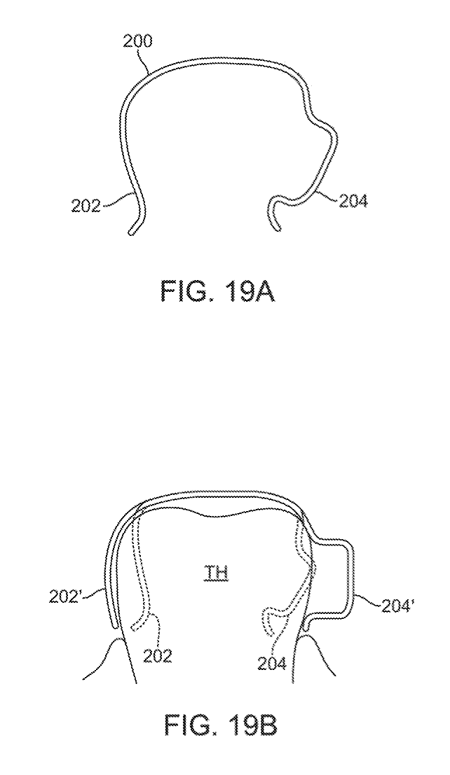

In yet another variation, FIGS. 19A and 19B illustrate an oral appliance 200 which may be pre-formed from a shape memory polymer or alloy or a superelastic material such as a Nickel-Titanium alloy, e.g., Nitinol. FIG. 19A shows oral appliance 200 in a first configuration where members 202, 204 are in an unbiased memory con figuration. When placed upon or against the tooth TH, members 202, 204 may be deflected into a second configuration where members 202′, 204′ are deformed to engage tooth TH in a secure interference fit, as shown in FIG. 19B. The biased member 204′ may be utilized to press the electronics and/or transducer assembly contained therein against the tooth surface as well as to maintain securement of the oral appliance 200 upon the tooth TH.

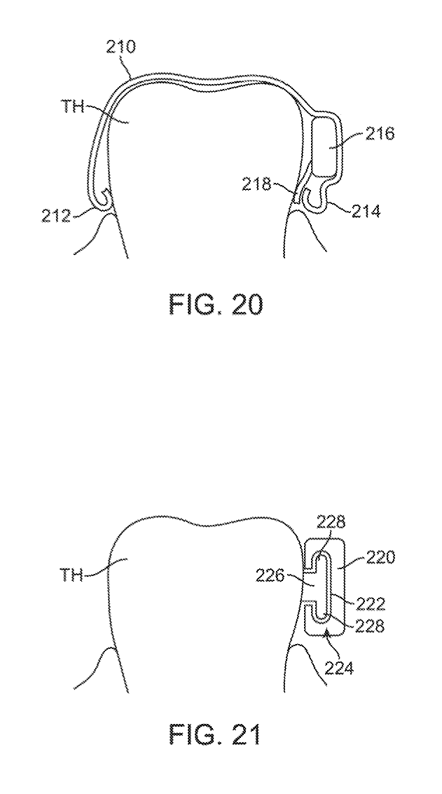

Similarly, as shown in FIG. 20, removable oral appliance 210 may have biased members to secure engage the tooth TH, as above. In this variation, the ends of the members 212, 214 may be configured into curved portions under which a transducer element 218 coupled to electronics assembly 216 may be wedged or otherwise secured to ensure mechanical contact against the tooth surface.

FIG. 21 shows yet another variation in which the oral appliance is omitted entirely. Here, a composite dental anchor or bracket 226, as described above, may be adhered directly onto the tooth surface. Alternatively, bracket 226 may be comprised of a biocompatible material, e.g., stainless steel, Nickel-Titanium, Nickel, ceramics, composites, etc., formed into a bracket and anchored onto the tooth surface. The bracket 226 may be configured to have a shape 228 over which an electronics and/or transducer assembly 220 may be slid over or upon via a channel 222 having a corresponding receiving configuration 224 for engagement with bracket 226. In this manner, assembly 220 may be directly engaged against bracket 226, through which a transducer may directly vibrate into the underlying tooth TH. Additionally, in the event that assembly 220 is removed from the tooth TH, assembly 220 may be simply slid or rotated off bracket 226 and a replacement assembly may be put in its place upon bracket 226.

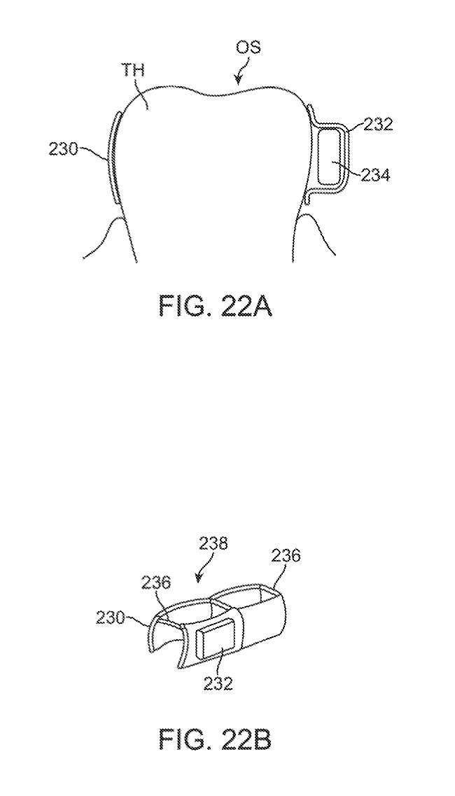

FIGS. 22A and 228 show partial cross-sectional side and perspective views, respectively, of yet another variation of an oral appliance 230. In this variation, the oral appliance 230 may be configured to omit an occlusal surface portion of the oral appliance 230 and instead engages the side surfaces of the tooth TH, such as the lingual and buccal surfaces only. The electronics and/or transducer assembly 234 may be contained, as above, within a housing 232 for contact against the tooth surface. Additionally, as shown in FIG. 22B, one or more optional cross-members 236 may connect the side portions of the oral appliance 230 to provide some structural stability when placed upon the tooth. This variation may define an occlusal surface opening 238 such that when placed upon the tooth, the user may freely bite down directly upon the natural occlusal surface of the tooth unobstructed by the oral appliance device, thereby providing for enhanced comfort to the user.

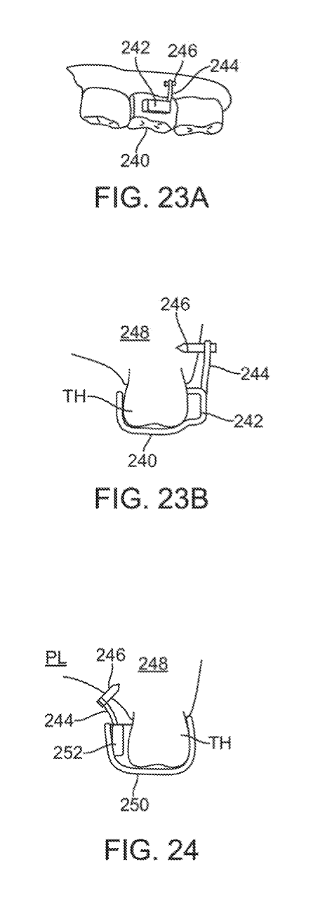

In yet other variations, vibrations may be transmitted directly into the underlying bone or tissue structures rather than transmitting directly through the tooth or teeth of the user. As shown in FIG. 23A, an oral appliance 240 is illustrated positioned upon the user's tooth, in this example upon a molar located along the upper row of teeth. The electronics and/or transducer assembly 242 is shown as being located along the buccal surface of the tooth. Rather than utilizing a transducer in contact with the tooth surface, a conduction transmission member 244, such as a rigid or solid metallic member, may be coupled to the transducer in assembly 242 and extend from oral appliance 240 to a post or screw 246 which is implanted directly into the underlying bone 248, such as the maxillary bone, as shown in the partial cross-sectional view of FIG. 23B. As the distal end of transmission member 244 is coupled directly to post or screw 246, the vibrations generated by the transducer may be transmitted through transmission member 244 and directly into post or screw 246, which in turn transmits the vibrations directly into and through the bone 248 for transmission to the user's inner ear.

FIG. 24 illustrates a partial cross-sectional view of an oral appliance 250 placed upon the user's tooth TH with the electronics and/or transducer assembly 252 located along the lingual surface of the tooth. Similarly, the vibrations may be transmitted through the conduction transmission member 244 and directly into post or screw 246, which in this example is implanted into the palatine one PL. Other variations may utilize this arrangement located along the lower row of teeth for transmission to a post or screw 246 drilled into the mandibular bone.

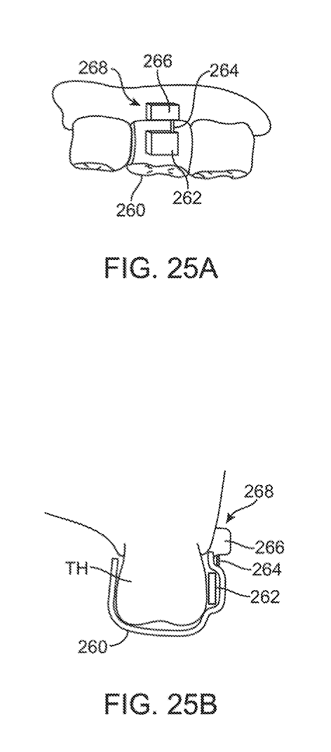

In yet another variation, rather utilizing a post or screw drilled into the underlying bone itself, a transducer may be attached, coupled, or otherwise adhered directly to the gingival tissue surface adjacent to the teeth. As shown in FIGS. 25A and 25B, an oral appliance 260 may have an electronics assembly 262 positioned along its side with an electrical wire 264 extending therefrom to a transducer assembly 266 attached to the gingival tissue surface 268 next to the tooth TH. Transducer assembly 266 may be attached to the tissue surface 268 via an adhesive, structural support arm extending from oral appliance 260, a dental screw or post, or any other structural mechanism. In use, the transducer may vibrate and transmit directly into the underlying gingival tissue, which may conduct the signals to the underlying bone.

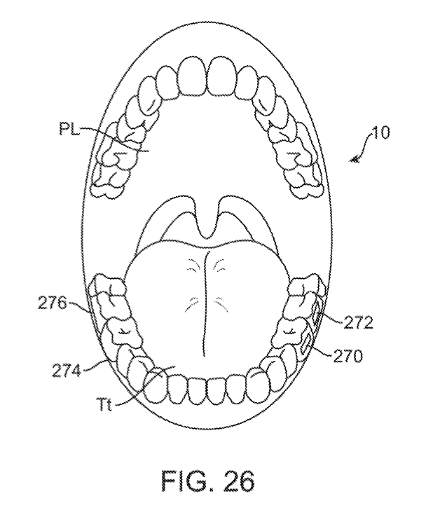

For any of the variations described above, they may be utilized as a single device or in combination with any other variation herein, as practicable, to achieve the desired hearing level in the user. Moreover, more than one oral appliance device and electronics and/or transducer assemblies may be utilized at any one time. For example, FIG. 26 illustrates one example where multiple transducer assemblies 270, 272, 274, 276 may be placed on multiple teeth. Although shown on the lower row of teeth, multiple assemblies may alternatively be positioned and located along the upper row of teeth or both rows as well. Moreover, each of the assemblies may be configured to transmit vibrations within a uniform frequency range. Alternatively in other variations, different assemblies may be configured to vibrate within non-overlapping frequency ranges between each assembly. As mentioned above, each transducer 270, 272, 274, 276 can be programmed or preset for a different frequency response such that each transducer may be optimized for a different frequency response and/or transmission to deliver a relatively high-fidelity sound to the user.

Moreover, each of the different transducers 270, 272, 274, 276 can also be programmed to vibrate in a manner which indicates the directionality of sound received by the microphone worn by the user. For example, different transducers positioned at different locations within the user's mouth can vibrate in a specified manner by providing sound or vibrational queues to inform the user which direction a sound was detected relative to an orientation of the user. For instance, a first transducer located, e.g., on a user's left tooth, can be programmed to vibrate for sound detected originating from the user's left side. Similarly, a second transducer located, e.g., on a user's right tooth, can be programmed to vibrate for sound detected originating from the user's right side. Other variations and queues may be utilized as these examples are intended to be illustrative of potential variations.

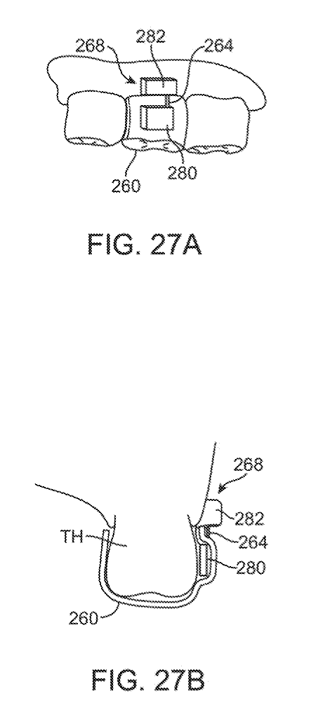

In variations where the one or more microphones are positioned in intra-buccal locations, the microphone may be integrated directly into the electronics and/or transducer assembly, as described above. However, in additional variation, the microphone unit may be positioned at a distance from the transducer assemblies to minimize feedback. In one example, similar to a variation shown above, microphone unit 282 may be separated from electronics and/or transducer assembly 280, as shown in FIGS. 27A and 27B. In such a variation, the microphone unit 282 positioned upon or adjacent to the gingival surface 268 may be electrically connected via wire(s) 264.

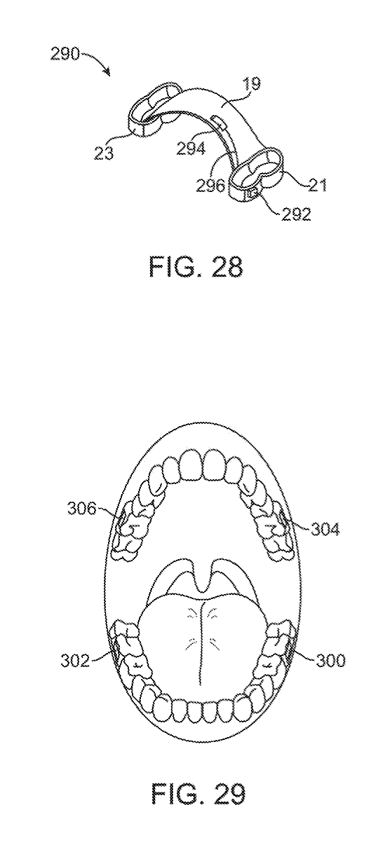

Although the variation illustrates the microphone unit 282 placed adjacent to the gingival tissue 268, unit 282 may be positioned upon another tooth or another location within the mouth. For instance, FIG. 28 illustrates another variation 290 which utilizes an arch 19 connecting one or more tooth retaining portions 21, 23, as described above. However, in this variation, the microphone unit 294 may be integrated within or upon the arch 19 separated from the transducer assembly 292. One or more wires 296 routed through arch 19 may electrically connect the microphone unit 294 to the assembly 292. Alternatively, rather than utilizing a wire 296, microphone unit 294 and assembly 292 may be wirelessly coupled to one another, as described above.

In yet another variation for separating the microphone from the transducer assembly. FIG. 29 illustrates another variation where at least one microphone 302 (or optionally any number of additional microphones 304, 306) may be positioned within the mouth of the user while physically separated from the electronics and/or transducer assembly 300. In this manner, the one or optionally more microphones 302, 304, 306 may be wirelessly coupled to the electronics and/or transducer assembly 300 in a manner which attenuates or eliminates feedback, if present, from the transducer.

The applications of the devices and methods discussed above are not limited to the treatment of hearing loss but may include any number of further treatment applications. Moreover, such devices and methods may be applied to other treatment sites within the body. Modification of the above-described assemblies and methods for caring out the invention, combinations between different variations as practicable, and variations of aspects of the invention that are obvious to those of skill in the art are intended to be within the scope of the claims.

What is claimed is:

1. A method for determining a pulmonary function of a patient, comprising:
a. mounting one or more sensors intra-orally upon at least one tooth via an intra-oral appliance, wherein the appliance produces an interference fit between the appliance and at least two surfaces of at least one tooth;
b. capturing intra-oral data via the one or more sensors; and
c. determining the pulmonary function based on an analysis of the intra-oral data.

2. The method of claim 1, further comprising determining an intermittent breathing condition from an intra-oral sound captured by the one or more sensors or determining a snoring condition from the intra-oral sound.

3. The method of claim 1, wherein mounting further comprises providing the appliance having an actuatable transducer disposed Within or upon a housing of the appliance.

4. The method of claim 3, wherein after determining the pulmonary function, maintaining contact between a surface of the at least one tooth and the actuatable transducer such that the transducer transmits vibrations to a surface of the at least one tooth.

5. The method of claim 1, wherein capturing comprises:
a. measuring a magnitude and a frequency of an intra-oral sound; and
b. determining one or more intervals between breaths from the intra-oral sound.

6. The method of claim 1, wherein capturing comprises measuring oxygen concentration or carbon dioxide saturation.

7. The method of claim 1, wherein capturing comprises measuring oxygen data through a lax stratum corneum or a dermal structure.

8. The method of claim 1, wherein capturing comprises performing, a dual-color ratiometric oxygen saturation measurement.

9. The method of claim 1, wherein capturing comprises measuring breath oxygen or carbon dioxide content.

10. The method of claim 1, wherein capturing comprises measuring inhaled and exhaled air for oxygen and/or carbon dioxide content.

11. The method of claim 1, further comprising providing a stimulus signal to a patient based on the pulmonary function.

12. The method of claim 11, further comprising applying the stimulus signal to a jaw.

13. The method of claim 11, further comprising generating a sensation comprising one or more of: sound, vibration and electrical stimulation.

14. The method of claim 11, further comprising altering a depth of sleep through the stimulus signal.

15. The method of claim 11, further comprising altering a body position through the stimulus signal.

16. The method of claim 1, wherein capturing comprises measuring cardiac signals.

17. The method of claim 16, wherein measuring cardiac signals comprises measuring EKG signals or ECG signals.

18. The method of claim 16, further comprising generating an alarm based on the cardiac signals.

19. The method of claim 1, further comprising releasing a drug from the appliance.

20. The method of claim 1, wherein the intra-oral appliance comprises a custom appliance.

21. The method of claim 20, wherein the one or more sensors comprise one of temperature sensors, flow velocity sensors, acoustic sensors, heart rate sensors, optical sensors, arterial tone sensors, oxygen sensors, EEG sensors, EKG sensors, pH sensors, or snoring sound sensors.

22. The method of claim 1, further comprising detecting one of: a sleep apnea condition, a snoring condition, a pulmonary condition and a bruxing condition, based upon the pulmonary function.

23. The method of claim 1, further comprising treating one of: a sleep apnea condition, a snoring condition, a pulmonary condition, and a bruxing condition.

24. The method of claim 1, further comprising, providing, therapy to the patient based upon the pulmonary function.

25. The method of claim 24, further comprising delivering a vibration on a tooth or a gum.

26. The method of claim 24, further comprising waking a patient.

27. The method of claim 24, further comprising delivering sound to wake a patient.

28. The method of claim 24, further comprising delivering electrical energy to stimulate nerves.

29. An apparatus for transmitting vibrations via at least one tooth to facilitate communications, comprising:
a housing having a shape which is conformable to at least a portion of the at least one tooth;
an actuatable transducer disposed within or upon the housing and in vibratory communication with a surface of the at least one tooth; and
a pulmonary detector coupled to the transducer.

30. The apparatus of claim 29, wherein the housing comprises an oral appliance having a shape which conforms to the at least one tooth.

31. The apparatus of claim 29, wherein the housing comprises a custom removable intra-oral appliance.

32. The apparatus of claim 29, wherein the housing is secured to a tooth or a mandible using one of a screw, an adhesive, a fastener, a suction cup, a Velcro mount.
